(19) United States
(12) Patent Application Publication
(10) Pub. No.: US 2005/0209639 A1
(43) Pub. Date: Sep. 22, 2005

(54) Fused suture knot

(75) Inventors: Suresh Gidwani, Watertown, MA (US); Yem Chin, Natick, MA (US)

Correspondence Address: HOFFMANN & BARON, LLP, 6900 JERICHO TURNPIKE, SYOSSET, NY, 11791, US

(73) Assignee: Boston Scientific Scimed, Inc.

(21) Appl. No.: 11/073,779

(22) Filed: Mar. 7, 2005

Related U.S. Application Data
(60) Provisional application No. 60/551,141, filed on Mar. 8, 2004.

(51) Int. Cl. A61B 17/04

(52) U.S. Cl. 606/228

(57) ABSTRACT

A securement location on a polymer thread structure includes the tying of a knot. In order to secure the knot, the knot is ultrasonically welded. Multifilament suture knots may be welded. The ultrasonic welding technique also tends to decrease the diameter of the knot from its pre-fused diameter. The resultant fused knot exhibits increased tensile and peel strength.

### CROSS-REFERENCE TO RELATED APPLICATIONS

This application claims benefit of priority of U.S. Provisional Application No. 60/551,141, filed Mar. 8, 2004.

### FIELD OF INVENTION

The present invention relates generally to formation of a securement location in polymer thread structure. More particularly, the present invention relates to the fusing of a surgical suture knot.

### BACKGROUND OF THE INVENTION

Polymer sutures are widely used in surgical applications for purposes such as closing wounds, joining tissue and aiding in delivering and implanting medical devices within the body. In many such applications, a knot is placed in the suture. This knot could be for the purposes of tying off a suture, joining the ends of one or more sutures together or providing a loop which can be grasped by the surgeon for delivery and implantation purposes.

While the knot serves adequately, there are certain distinct disadvantages in knotted sutures. The strength of the knot is dependent upon an individual surgeon's technique. Also, especially with the use of synthetic polymer sutures, the knot may have a tendency to become loose or even untie. To overcome this, in certain applications, the surgeon may take the extra step of applying a glue or adhesive to the knot to prevent subsequent loosening. As may be appreciated, formation of a knot in a suture, especially in situations where the knot is subsequently glued, provides an area of increased thickness. This may lead to certain patient discomfort and an increase in healing time of the site. Moreover, gluing is not always effective in securing the knot. In situations where the suture may contain certain polymers, such as polytetrafluoroethylene (PTFE), the knot is generally resistant to gluing.

The art has looked to alternatives to knotting sutures. One known technique is to join suture ends by processes such as fusing, melting or welding the ends together. In particular, use of ultrasonic energy has been used extensively to weld suture ends together without the need for knotting.

Ultrasonic suture welding devices are well known in the art. On example is shown in U.S. Pat. No. 5,893,880. Overlapped portions of a monofilament suture are placed between the opposed surfaces of an ultrasonic welding tip. By ultrasonic energy, the overlapped portions are fused together. The fused weld joint of this type exhibits desirably high tensile strength.

More specifically, in U.S. Pat. No. 6,409,743 an ultrasonic welding tool is shown. The tool further employs a fusable polymeric sleeve which supports therein overlapped portions of the monofilament sutures. Ultrasonic energy is applied to the sleeve and the sleeve and the overlapped portions of the sutures are fused together. Conventional lap joints position suture ends in overlapping nature, such that they lay on top of one another, both sharing a common plane along the overlapping portion. Since many sutures are actually generally round in cross-section, when their ends are placed on top of each other to form the lap, their respective surfaces may be said to be touching along a common tangent. Fusion of the lap by welding occurs at this tangent. A conventional lap weld is shown in FIG. 7.

While the ultrasonic fusing of sutures has been successfully employed, such fusion is not without distinct disadvantages. It has been found that while conventional fused lap joints may exhibit, in some instances, sufficient tensile strength, the lap joints suffer from undesirably low peel or tear strength. This could result in joint failure, particularly once a peel line is initiated. Propagation along the peel line generally takes less energy than the initial tear and consequently it is important to prevent the tear from being initiated.

Attempts have been made to provide improved welded joints. For example, in U.S. Pat. No. 3,657,056 a suture joint is formed by knotting the ends of a suture and then ultrasonically welding the knot itself. In this manner there is an attempt to obtain the benefits of both securement techniques, i.e., the peel or tear strength of a knot, with the long-term benefits of an ultrasonic weld. However, as may be appreciated, the conventional ultrasonic welding of a conventionally formed knot does nothing to decrease the size of the knot placed in the suture. Thus, ultrasonically welded knots still exhibit a large profile which reduces their desirability, both for smoothness in delivery and deployment in the body, as well as patient comfort once implanted.

Additionally, ultrasonic welding of sutures has been found to be viable only with respect to monofilament sutures. The solid surface of the monofilament provides sufficient surface contact between the overlapped portions so as to effectively fuse the portions together. In surgical practice however, multifilament sutures, such as braided sutures, are preferred by surgeons over monofilament, as the multifilament sutures exhibit enhanced physical properties, such as, increased flexibility. Difficulties have arisen in attempts to ultrasonically weld multifilament sutures. The suture itself is formed of plural individual fibers which are braided or wrapped. This results in spaces between the individual fibers. In ultrasonic welding the spaces tend to dissipate the ultrasonic energy and therefore fail to adequately weld the overlapped portions together.

It is therefore desirable to provide a suture securement technique which exhibits high tensile and peel strength while being usable with both monofilament and multifilament sutures.

### SUMMARY OF THE INVENTION

The present invention provides a polymer thread structure having a knot formed therein defining a securement location. The knot is subsequently fused. The knot may be a formed intermediate an elongate thread or may be formed to tie together two ends of a single thread or the ends of two threads. The thread structure may be a multifilament thread which may be ultrasonically fused.

Moreover, subsequent to fusing, the knot exhibits a knot diameter which is less than the knot diameter prior to fusing.

In a method aspect of the present invention a method of forming a securement location in a polymer thread structure is provided. The method includes the steps of forming a knot in the thread structure and fusing the knot. The thread structure which may be a multifilament thread is preferably ultrasonically welded.

The welding of the knot decreases the knot diameter.

### BRIEF DESCRIPTION OF THE DRAWINGS

FIG. 1 shows by way of an example a schematic representation of the cross-section of a multifilament suture.

FIG. 2 shows a conventional square knot tied in a suture loop.

FIGS. 3A-3L show variations of knots which may be used in sutures.

FIG. 4 shows the knot of FIG. 1 after ultrasonic welding.

FIG. 5 shows schematically a weld base and horn used for ultrasonic welding.

FIG. 6 shows samples of weld energy wave shapes.

FIG. 7 shows a prior art lap weld.

### DETAILED DESCRIPTION OF THE INVENTION

The present invention provides for the formation of a securement location within a thread structure. As used herein the term securement location refers generally to a knot which may be placed in a suture. The knot may be used to join the ends of single suture or the ends of two different sutures. In addition, the securement location may be a knot placed centrally in the suture which may be used to provide a loop which may be grasped by a surgeon for delivering and implanting of implantable medical devices. The present invention finds particular utility with multifilament sutures. Moreover, the term thread structure refers to one or more threads.

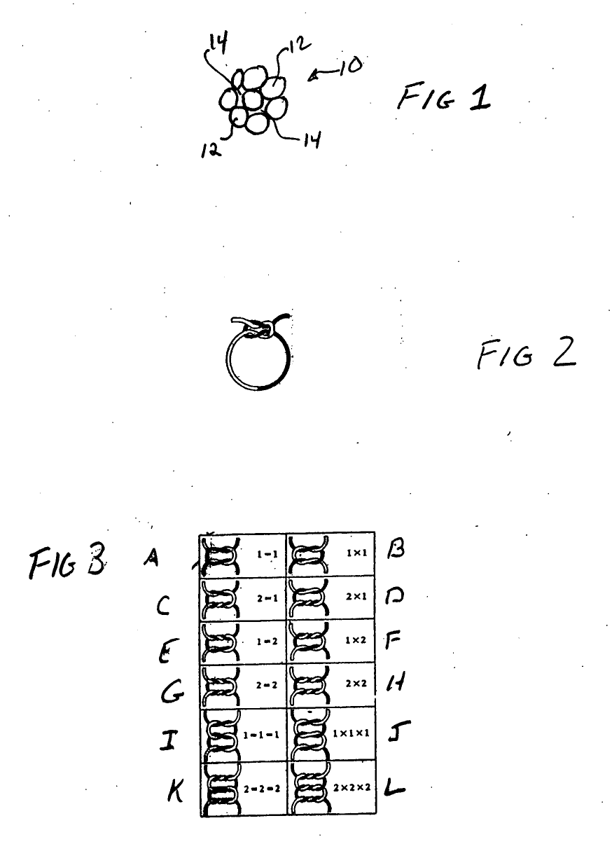

Referring now to FIG. 1, a schematic representation of the cross-section of a multifilament suture is shown. Suture 10 includes a plurality of individual fibers 12 joined together to form a suture. Various constructions of multifilaments are known such as, for example, braided sutures. As the multifilament suture is formed of individually joined filaments, spaces 14 occur between the individual filaments. Heretofore, it has been found difficult to ultrasonically weld multifilaments due to these spaces, inasmuch as the spaces tend to dissipate the ultrasonic energy creating an ineffective joint. Moreover, many of the multifilament sutures are synthetic, formed from polytetrafluoroethylene (PTFE) impregnated polyester. Synthetics of this type, due to the known properties of PTFE, are not susceptible to secure knot tying as the knot has a tendency to slip. Other polymeric materials, such as nylons and other polyamide materials also have a tendency to slip when knotted. Even where additional securement techniques like gluing is employed, the knot is not satisfactory as PTFE has a tendency not to accept the glue.

Among the useful structures are multifilament sutures or threads which include a monofilament core. Multifilament threads may be combined into twisted, braided or other configurations for use. A wide variety of thermoplastic polymers may be employed including polyesters, fluorinated polymers, nylons, polyamides and copolymers and combinations thereof.

The present invention provides a technique for placing a securement location within such synthetic multifilament sutures employing conventional ultrasonic welding techniques.

It is well known to ultrasonically weld the overlapped portions of monofilament sutures. Reference is made to U.S. Pat. Nos. 5,893,880 issued Apr. 13, 1999 entitled “Fused Loop Filamentous Material”; and U.S. Pat. No. 6,409,743 issued Jun. 25, 2002 entitled “Device and Methods for Securing Sutures and Ligatures Without Knots”, which show conventional techniques for ultrasonic welding monofilament sutures. Each of these patents is incorporated by reference herein for all purposes. The present invention employs such conventional techniques to ultrasonically weld a knot placed in synthetic multifilament sutures.

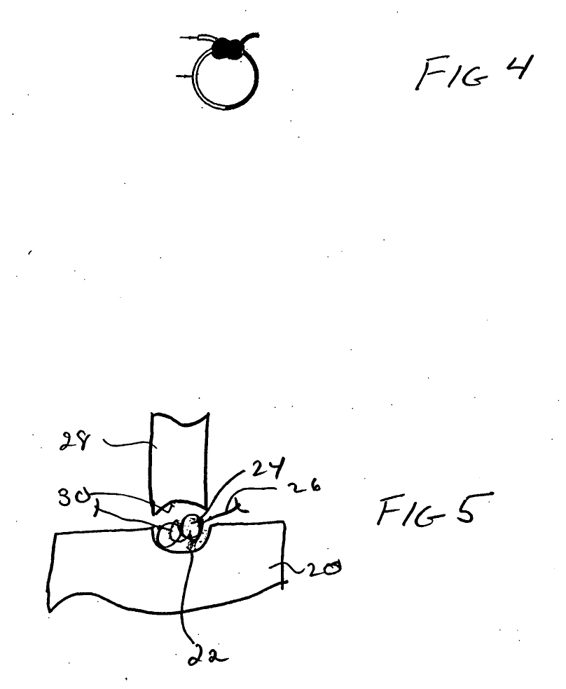

Referring now to FIG. 2, a multifilament structure such as the type shown in FIG. 1 is shown in a conventional square knotted configuration. The depiction of FIG. 2 represents a knot placed in a single suture or for joining the ends of a single or two adjoining sutures. While a square knot is shown in FIG. 2, it may be appreciated that other conventional knotting techniques such as those shown in FIGS. 3A-L may also be employed.

The knot is ultrasonically welded employing the techniques and equipment shown in the above-referenced incorporated patents, as discussed below. Knots of the present invention may be used on a wide variety of medical devices, including implantable devices, delivery devices and devices used on the body. The inventive knot structure can be used to make another knot, such as a slip knot more secure.

A variety of wave shapes of energy can be used to form the weld. FIG. 6 shows such shapes. Compression pressure can be added and varied to achieve desired results. Multiple short bursts of energy, a long burst of energy or a combination thereof may be used in fusing or welding.

Any useful form of energy which effectively fuses the structure together may be employed provided it does not deleteriously effect the desired properties While ultrasonic energy has been found to be particularly effective in forming the weld, other forms of energy, which ultimately result in fusion of the filaments, such as electromagnetic energy and mechanical energy may be used. Radiation from RF, laser, UV light, infrared light are among the possible sources, as are heat, pressure or combinations of various fusion or welding energy sources.

### EXAMPLE

Examples were prepared of the ultrasonically welded multifilament knotted suture. The suture employed is USP 2-0 (Metric 3) Genzyme Code 113-T, Lot No. 612657, green braided polyester suture.

The ultrasonic welding tool described in the above-referenced patent employed was “Shoulder Fixation System”, Catalog No. 1104 from Axya Medical, Inc. A spring loaded welding disposal sleeve, Catalog No. 1258 (for USP 2-0 suture) of Axya Medical, Inc. was also employed. The specific knot tested was a 2=2 knot configuration as illustrated in FIG. 3G on green braided polyester suture USP 2-0. The knot was welded in conventional fashion as described in the above incorporated patents.

Four successfully welded knots were tested for loop tensile strength. The testing equipment employed includes Chilton Tensile 8E40973-10 ID No. 00829, including a J-hook attachment on Chilton Gauge and a J-hook to pull the suture. The Chilton was attached to the workbench with a C clamp.

Each of the samples exhibited superior loop tensile strength as noted hereinbelow.

| Sample No. | Loop Tensile Strength |
|---|---|
| 1 | 22.0 lbs. |
| 2 | 13.5 lbs. |
| 3 | 16.0 lbs. |
| 4 | 16.5 lbs. |

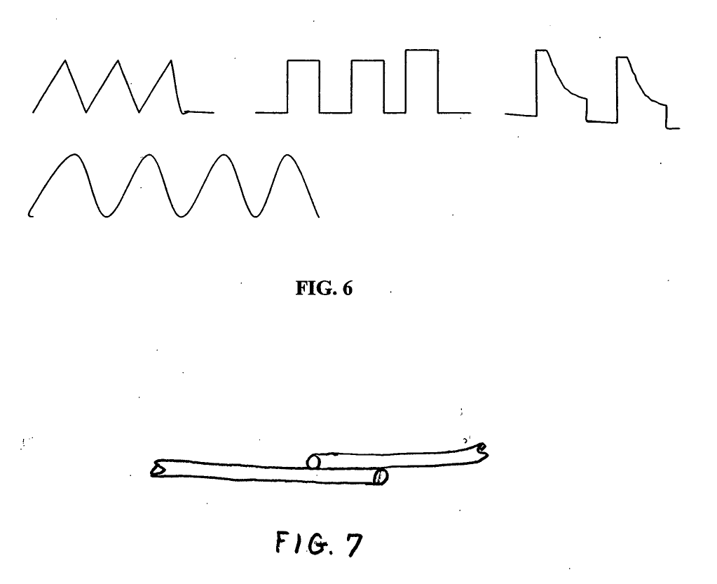

The above results were compared with a conventional lap weld, that is a weld of the multifilament suture ends layered one over the other such that they share a common plane along the overlapped portion, i.e., without placing a knot in the suture (FIG. 7). Such a lap weld has a tensile strength of 2.0 lbs. Also the above results were compared with a non-welded square knot which exhibited a tensile strength of 5.5 lbs. As can be appreciated the welded knot of the present invention exhibited superior tensile strength as compared with both a welded lap weld and a non-welded knot.

During sample testing, several knots slipped during the ultrasonic welding process. As a result, as shown in FIG. 5, a modified nest or sleeve was designed so as to securely hold the knot in place.

FIG. 5 shows a weld base 20 having a cavity 22 centrally formed therein which accommodates the knot 24 of suture 26. A weld horn 28 having a recess 30 therein is engagable with the weld base 20. The cavity 22 in the weld base as well as the recess 30 in the weld horn 28 form an area for securely holding the knot 24 preventing knot slippage during welding. This allowed the knot to maintain its tight configuration during welding.

Moreover, it is contemplated that modifications to the weld base and the horn may be provided so as to change the profile of the knot after ultrasonic welding. This change in profile may reduce the shape, size or diameter of the knot from its pre-welded configuration shown in FIGS. 2 and 3 to a post-welded configuration shown in FIG. 4. Reduction in the knot profile is particularly useful for lessening of patient discomfort and a decrease in healing times.

In another aspect of the invention, the knot is formed from a mono-, multifilament or a combination of mono- and multifilament sutures. This knot profile reduction is also seen in monofilament sutures such as nylon monofilaments, and in combinations of mono- and multifilament sutures.

Various changes to the foregoing described and shown structures would now be evident to those skilled in the art. Accordingly, the particularly disclosed scope of the invention is set forth in the following claims.

1. A multifilament polymer thread structure having an ultrasonically fused knot formed therein.

2. A multifilament polymer thread structure of claim 1 wherein said thread structure includes an elongate thread having said knot formed intermediate ends thereof.

3. A multifilament polymer thread structure of claim 1 wherein said threaded structure includes an elongate thread having ends thereofjoined to form said knot.

4. A multifilament polymer thread structure of claim 1 wherein said thread structure includes two threads having ends thereof joined to form said knot.

5. A multifilament polymer thread structure of claim 1 wherein said thread structure is a surgical suture.

6. A multifilament polymer thread structure of claim 1 wherein said polymer thread structure comprises polyester.

7. A multifilament polymer thread structure of claim 6 wherein said polyester thread suture is impregnated with polytetrafluoroethylene.

8. A multifilament polymer thread structure of claim 1 wherein said polymer thread is selected from the group consisting of fluorinated polymers, nylon, polyamides and copolymers and combinations thereof.

9. A multifilament polymer thread structure of claim 1 wherein said thread strand is braided.

10. A multifilament polymer thread structure of claim 1 wherein said fused knot has a final diameter which is smaller than its diameter before said ultrasonic fusing.

11. A method of forming a securement location in a multifilament polymer thread structure comprising the step of:
forming a knot in said thread structure; and
ultrasonically fusing said knot.

12. A method of claim 11 wherein said forming step including:
providing an elongate thread; and
forming said knot intermediate the ends of said thread.

13. A method of claim 1 1 wherein said forming step includes;
providing an elongate thread; and
forming said knot by joining the ends thereof.

14. A method of claim 11 wherein said forming step includes;
providing two elongate threads; and
joining an end of one thread to an end of the other thread to form said knot.

15. A method of claim 11 wherein said thread structure is a surgical suture.

16. A method of claim 15 wherein said polymer thread structure is nylon.

17. A method of claim 11 wherein said fusing step includes:
ultrasonically welding said knot.

18. A method of claim 11 wherein said knot has a first diameter prior to said fusing step and a second diameter less than said first diameter after said fusing step.

19. A polymer thread structure having a knot formed therein, said knot being subsequently fused and having a fused knot diameter which is less than the diameter of the knot prior to being fused.

20. A polymer thread structure of claim 19 wherein said thread structure includes an elongate thread having said knot formed intermediate ends thereof.

21. A polymer thread structure of claim 19 wherein said threaded structure includes an elongate thread having ends thereof joined to form said knot.

22. A polymer thread structure of claim 19 wherein said thread structure includes two threads having ends thereof joined to form said knot.

23. A polymer thread structure of claim 19 wherein said thread structure is a surgical suture.

24. A polymer thread structure of claim 19 wherein said polymer thread structure is multifilament.

25. A polymer thread structure of claim 19 wherein said polymer thread structure is monofilament nylon.

26. A polymer thread structure of claim 19 wherein said knot is ultrasonically fused.

27. A method of forming a securement location in a polymer thread structure comprising the step of:
forming a knot in said thread structure, said knot having a knot diameter; and
fusing said knot, to decrease said knot diameter.

28. A method of claim 27 wherein said forming step including:
providing an elongate thread; and
forming said knot intermediate the ends of said thread.

29. A method of claim 27 wherein said forming step includes;
providing an elongate thread; and
forming said knot by joining the ends thereof.

30. A method of claim 27 wherein said forming step includes;
providing two elongate threads; and
joining an end of one thread to an end of the other thread to form said knot.

31. A method of claim 27 wherein said thread structure is a surgical suture.

32. A method of claim 31 wherein said polymer thread structure is multifilament.

33. A method of claim 31 wherein said polymer thread structure is a monofilament nylon.

34. A method of claim 27 wherein said fusing step includes:
ultrasonically welding said knot.
